(19) United States
(12) Patent Application Publication
(10) Pub. No.: US 2005/0100508 A1
(43) Pub. Date: May 12, 2005

(54) Methods for identifying drug combinations for the treatment of proliferative diseases

(76) Inventors: M. James Nichols, Boston, MA (US); Margaret S. Lee, Middleton, MA (US); Curtis Keith, Boston, MA (US); Yanzhen Zhang, Sudbury, MA (US)

Correspondence Address: CLARK & ELBING LLP, 101 FEDERAL STREET, BOSTON, MA, 02110, US

(21) Appl. No.: 10/855,130

(22) Filed: May 27, 2004

Related U.S. Application Data
(60) Provisional application No. 60/519,551, filed on Nov. 12, 2003.

(51) Int. Cl. A61K 49/00; A61K 48/00; A61K 39/395; A61K 31/225

(52) U.S. Cl. 424/9.2; 514/44; 424/146.1

(57) ABSTRACT

The invention features methods for identifying new combination therapies for the treatment of cancer and other proliferative diseases.

### CROSS-REFERENCE TO RELATED APPLICATIONS

This application claims the benefit of U.S. Provisional Application No. 60/519,551, filed Nov. 12, 2003, hereby incorporated by reference.

### BACKGROUND OF THE INVENTION

The present invention relates to the treatment of cancer and other proliferative diseases.

Cancer is a disease marked by the uncontrolled growth of abnormal cells. Cancer cells have overcome the barriers imposed in normal cells, which have a finite lifespan, to grow indefinitely. As the growth of cancer cells continue, genetic alterations may persist until the cancerous cell has manifested itself to pursue a more aggressive growth phenotype. If left untreated, metastasis, the spread of cancer cells to distant areas of the body by way of the lymph system or bloodstream, may ensue, destroying healthy tissue.

The treatment of cancer has been hampered by the fact that there is considerable heterogeneity even within one type of cancer. Some cancers, for example, have the ability to invade tissues and display an aggressive course of growth characterized by metastases. These tumors generally are associated with a poor outcome for the patient. Ultimately, tumor heterogeneity results in the phenomenon of multiple drug resistance, i.e., resistance to a wide range of structurally unrelated cytotoxic anticancer compounds, J. H. Gerlach et al., Cancer Surveys, 5:25-46 (1986). The underlying cause of progressive drug resistance may be due to a small population of drug-resistant cells within the tumor (e.g., mutant cells) at the time of diagnosis, as described, for example, by J. H. Goldie and Andrew J. Coldman, Cancer Research, 44:3643-3653 (1984). Treating such a tumor with a single drug can result in remission, where the tumor shrinks in size as a result of the killing of the predominant drug-sensitive cells. However, with the drug-sensitive cells gone, the remaining drug-resistant cells can continue to multiply and eventually dominate the cell population of the tumor. Therefore, the problems of why metastatic cancers develop pleiotropic resistance to all available therapies, and how this might be countered, are the most pressing in cancer chemotherapy.

Anticancer therapeutic approaches are needed that are reliable for a wide variety of tumor types, and particularly suitable for invasive tumors. Importantly, the treatment must be effective with minimal host toxicity. In spite of the long history of using multiple drug combinations for the treatment of cancer and, in particular, the treatment of multiple drug resistant cancer, positive results obtained using combination therapy are still frequently unpredictable.

### SUMMARY OF THE INVENTION

The invention features methods for identifying new combination therapies for the treatment of cancer and other proliferative diseases.

In a first aspect, the invention features a method for identifying a combination that may be useful for the treatment of a proliferative disease. In this method, proliferating cells (e.g., cancer cells or a cancer cell line) are contacted in vitro with (i) an agent that reduces mitotic kinesin biological activity and (ii) a candidate compound. Using any acceptable assay, it is then determined whether the combination of the agent and the candidate compound reduces cell proliferation, relative to proliferation of cells contacted with the agent but not contacted with the candidate compound. A reduction in cell proliferation identifies the combination as a combination that may be useful for the treatment of a proliferative disease.

In another aspect, the invention features another method for identifying a combination that may be useful for the treatment of a proliferative disease. This method includes the steps of (a) identifying a compound that reduces protein tyrosine phosphatase biological activity; (b) contacting proliferating cells in vitro with an agent that reduces mitotic kinesin biological activity and the compound identified in step (a); and (c) determining whether the combination of the agent and the compound identified in step (a) reduces cell proliferation, relative to proliferation of cells contacted with the agent but not contacted with the compound identified in step (a) or contacted with the compound identified in step (a) but not contacted with the agent. A reduction in cell proliferation identifies the combination as a combination that may be useful for the treatment of a proliferative disease.

In either of the foregoing aspects, the agent that reduces mitotic kinesin biological activity may be, for example, a mitotic kinesin inhibitor, an antisense compound or RNAi compound that reduces the expression levels of a mitotic kinesin, a dominant negative mitotic kinesin, an expression vector encoding such a dominant negative mitotic kinesin, an antibody that binds a mitotic kinesin and reduces mitotic kinesin biological activity, or an aurora kinase inhibitor. Desirably, the agent that reduces mitotic kinesin biological activity reduces the biological activity of HsEg5/KSP. Exemplary mitotic kinesin biological activities are enzymatic activity, motor activity, and binding activity.

In still another aspect, the invention features another method for identifying a compound that may be useful for the treatment of a proliferative disease. This method includes the steps of: (a) providing proliferating cells engineered to have reduced mitotic kinesin biological activity; (b) contacting the cells with a candidate compound; and (c) determining whether the candidate compound reduces cell proliferation, relative to cells not contacted with the candidate compound. A reduction in cell proliferation identifies the compound as a compound that may be useful for the treatment of a proliferative disease.

In another aspect, the invention features yet another method for identifying a combination that may be useful for the treatment of a proliferative disease. This method includes the steps of: (a) contacting proliferating cells in vitro with an agent that reduces protein tyrosine phosphatase biological activity and a candidate compound; and (b) determining whether the combination of the agent and the candidate compound reduces cell proliferation, relative to proliferation of cells contacted with the agent but not contacted with the candidate compound. A reduction in cell proliferation identifies the combination as a combination that may be useful for the treatment of a proliferative disease.

In a related aspect, the invention features a method for identifying a combination that may be useful for the treatment of a proliferative disease. This method includes the steps of: (a) identifying a compound that reduces mitotic kinesin biological activity; (b) contacting proliferating cells in vitro with an agent that reduces protein tyrosine phosphatase biological activity and the compound identified in step (a); and (c) determining whether the combination of the agent and the compound identified in step (a) reduces cell proliferation, relative to proliferation of cells contacted with the agent but not contacted with the compound identified in step (a) or contacted with the compound identified in step (a) but not contacted with the agent. A reduction in cell proliferation identifies the combination as a combination that may be useful for the treatment of a proliferative disease.

In either of the foregoing aspects, the agent that reduces protein tyrosine phosphatase biological activity is a protein tyrosine phosphatase inhibitor, an antisense compound or RNAi compound that reduces the expression levels of a protein tyrosine phosphatase, a dominant negative protein tyrosine phosphatase, an expression vector encoding said dominant negative protein tyrosine phosphatase, an antibody that binds a protein tyrosine phosphatase and reduces protein tyrosine phosphatase biological activity, or a farnesyltransferase inhibitor. Desirably, the agent reduces the biological activity of a protein tyrosine phosphatase selected from PTP1B, PRL-1, PRL-2, PRL-3, SHP-1, SHP-2, MKP-1, MKP-2, CDC14, CDC25A, CDC25B, and CDC25C.

In another aspect, the invention features another method for identifying a compound that may be useful for the treatment of a proliferative disease. This method includes the steps of: (a) providing proliferating cells engineered to have reduced protein tyrosine phosphatase biological activity; (b) contacting the cells with a candidate compound; and (c) determining whether the candidate compound reduces cell proliferation, relative to cells not contacted with the candidate compound. A reduction in cell proliferation identifies the compound as a compound that may be useful for the treatment of a proliferative disease.

In any of the foregoing aspect, the cells are desirably cancer cells or cells from a cancer cell line.

By “more effective” is meant that a method, composition, or kit exhibits greater efficacy, is less toxic, safer, more convenient, better tolerated, or less expensive, or provides more treatment satisfaction than another method, composition, or kit with which it is being compared. Efficacy may be measured by a skilled practitioner using any standard method that is appropriate for a given indication.

By “mitotic kinesin inhibitor” is meant an agent that binds a mitotic kinesin and reduces, by a significant amount (e.g., by at least 10%, 20% 30% or more), the biological activity of that mitotic kinesin. Mitotic kinesin biological activities include enzymatic activity (e.g., ATPase activity), motor activity (e.g., generation of force) and binding activity (e.g., binding of the motor to either microtubules or its cargo).

By “dominant negative” is meant a protein that contains at least one mutation that inactivates its physiological activity such that the expression of this mutant in the presence of the normal or wild-type copy of the protein results in inactivation of or reduction of the activity of the normal copy. Thus, the activity of the mutant “dominates” over the activity of the normal copy such that even though the normal copy is present, biological function is reduced. In one example, a dimer of two copies of the protein are required so that even if one normal and one mutated copy are present there is no activity; another example is when the mutant binds to or “soaks up” other proteins that are critical for the function of the normal copy such that not enough of these other proteins are present for activity of the normal copy.

By “protein tyrosine phosphatase” or “PTPase” is meant an enzyme that dephosphorylates a tyrosine residue on a protein substrate.

By “protein tyrosine phosphatase inhibitor” is an agent that binds a protein tyrosine phosphatase and inhibits (e.g. by at least 10%, 20%, or 30% or more) the biological activity of that protein tyrosine phosphatase.

By “dual specificity phosphatase” is meant a protein phosphatase that can dephosphorylate both a tyrosine residue and either a serine or threonine residue on the same protein substrate. Dual specificity phosphatases include MKP-1, MKP-2, and the cell division cycle phosphatase family (e.g., CDC14, CDC25A, CDC25B, and CDC25C). Dual specificity phosphatases are considered to be protein tyrosine phosphatases.

By “antiproliferative agent” is meant a compound that, individually, inhibits cell proliferation. Antiproliferative agents of the invention include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists and antagonists, endothelin A receptor antagonists, retinoic acid receptor agonists, immunomodulators, hormonal and antihormonal agents, photodynamic agents, and tyrosine kinase inhibitors.

By “inhibits cell proliferation” is meant measurably slows, stops, or reverses the growth rate of cells in vitro or in vivo. Desirably, a slowing of the growth rate is by at least 20%, 30%, 50%, 60%, 70%, 80%, or 90%, as determined using a suitable assay for determination of cell growth rates (e.g., a cell growth assay described herein). Typically, a reversal of growth rate is accomplished by initiating or accelerating necrotic or apoptotic mechanisms of cell death in the neoplastic cells.

By “a sufficient amount” is meant the amount of a compound, in a combination according to the invention, required to inhibit the growth of the cells of a neoplasm in vivo. The effective amount of active compound(s) used to practice the present invention for therapeutic treatment of proliferative diseases (i.e., cancer) varies depending upon the manner of administration, the age, race, gender, organ affected, body weight, and general health of the subject. Ultimately, the attending physician or veterinarian will decide the appropriate amount and dosage regimen.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition.

By a “high dosage” is meant at least 5% (e.g., at least 10%, 20%, 50%, 100%, 200%, or even 300%) more than the highest standard recommended dosage of a particular compound for treatment of any human disease or condition.

The phrase “pharmaceutically acceptable” refers to molecular entities and compositions that do not produce an adverse, allergic or other untoward reaction when administered to patient.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art.

By “patient” is meant any animal (e.g., a human). Non-human animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

Compounds useful in the invention include those described herein in any of their pharmaceutically acceptable forms, including isomers such as diastereomers and enantiomers, salts, solvates, and polymorphs, thereof, as well as racemic mixtures of the compounds described herein.

Other features and advantages of the invention will be apparent from the following detailed description, and from the claims.

### DETAILED DESCRIPTION

The invention features methods for the identification of combination therapies for the treatment of proliferative disorders.

Normal cells have signaling mechanisms that regulate growth, mitosis, differentiation, cell function, and cell death in a programmed fashion. Defects in the signaling pathways that regulate these functions can result in uncontrolled growth and proliferation, which can manifest as cancer, hyperplasias, restenosis, cardiac hypertrophy, immune disorders and inflammatory disorders.

Mitotic kinesins are essential motors in mitosis. They control spindle assembly and maintenance, attachment and proper positioning of the chromosomes to the spindle, establish the bipolar spindle and maintain forces in the spindle to allow movement of chromosomes toward opposite poles. Perturbations of mitotic kinesin function cause malformation or dysfunction of the mitotic spindle, frequently resulting in cell cycle arrest and cell death.

Protein tyrosine phosphatases (PTPases) are intracellular signaling molecules that dephosphorylate a tyrosine residue on a protein substrate, thereby modulating certain cellular functions. In normal cells, they typically act in concert with protein tyrosine kinases to regulate signaling cascades through the phosphorylation of protein tyrosine residues. Phosphorylation and dephosphorylation of the tyrosine residues on proteins controls cell growth and proliferation, cell cycle progression, cytoskeletal integrity, differentiation and metabolism. In various metastatic and cancer cell lines, PTP1B and the family of Phosphatases of Regenerating Liver (PRL-1, PRL-2, and PRL-3) have been shown to be overexpressed. For example, PRL-3 (also known as PTP4A3) is expressed in relatively high levels in metatstatic colorectal cancers (Saha et al., Science 294: 1343-1346, 2001.). PRL-1 localizes to the mitotic spindle and is required for mitotic progression and chromosome segregation. PRL phosphatases promote cell migration, invasion, and metastasis, and inhibition of these PTPases has been shown to inhibit proliferation of cancer cells in vitro and tumors in animal models.

We previously demonstrated that the combination of chlorpromazine and pentamidine work in concert to reduce cell proliferation (U.S. Pat. No. 6,569,853). We now show that chlorpromazine acts as an inhibitor of mitotic kinesin. Pentamidine has been demonstrated to be an inhibitor of the PRL phosphatases (Pathak et al., Mol. Cancer Ther. 1: 1255-1264, 2002).

Based on the foregoing observations, we conclude that combinations of an agent that reduces the biological activity of a mitotic kinesin with an agent that reduces the activity of a protein tyrosine phosphatase are useful for reducing cell proliferation and, hence, for treating proliferative diseases.

### Mitotic Kinesins

Mitotic kinesins include HsEg5/KSP, KIFC3, CHO2, MKLP, MCAK, Kin2, Kif4, MPP1, CENP-E, NYREN62, LOC8464, and KIF8. Other mitotic kinesins are described in U.S. Pat. Nos. 6,414,121, 6,582,958, 6,544,766, 6,492,158, 6,455,293, 6,440,731, 6,437,115, 6,420,162, 6,399,346, 6,395,540, 6,383,796, 6,379,941, and 6,248,594. The GenBank Accession Nos. of representive mitotic kinesins are provided in Table 1.

TABLE 1
Human mitotic kinesins

| Protein name | GenBank Accession No. |
|---|---|
| Eg5/KSP | AA857025, U37426, X85137 |
| KIFC3 | BC001211 |
| MKLP1 | AI131325, AU133373, X67155 |
| MCAK | AL046197, U63743 |
| KIN2 | Y08319 |
| KIF4 | AF071592 |
| MPP1 | AL117496 |
| CENP-E | Z15005 |
| CHO2 | AL021366 |
| HsNYREN62 | AF155117 |
| HsLOC8464 | NM_032559 |
| KIF8 | AB001436 |

HsEg5/KSP has been cloned and characterized (see, e.g., Blangy et al., Cell, 83:1159-69 (1995); Galgio et al., J. Cell Biol., 135:399-414, 1996; Whitehead et al., J. Cell Sci., 111:2551-2561, 1998; Kaiser, et al., J. Biol. Chem., 274:18925-31, 1999; GenBank accession numbers: X85137, NM 004523). Drosophila (Heck et al., J. Cell Biol., 123:665-79, 1993) and Xenopus (Le Guellec et al., Mol. Cell Biol., 11:3395-8, 1991) homologs of KSP have been reported. Drosophila KLP61F/KRP130 has reportedly been purified in native form (Cole, et al., J. Biol. Chem., 269:22913-22916, 1994), expressed in E. coli, (Barton, et al., Mol. Biol. Cell, 6:1563-74, 1995) and reported to have motility and ATPase activities (Cole, et al., supra; Barton, et al., supra). Xenopus Eg5/KSP was expressed in E. coli and reported to possess motility activity (Sawin, et al., Nature, 359:540-3, 1992; Lockhart and Cross, Biochemistry, 35:2365-73, 1996; Crevel, et al, J. Mol. Biol., 273:160-170, 1997) and ATPase activity (Lockhart and Cross, supra; Crevel et al., supra).

Besides KSP, other members of the BimC family include BimC, CIN8, cut7, KIP1, KLP61F (Barton et al., Mol. Biol. Cell. 6:1563-1574, 1995; Cottingham et al., J. Cell Biol. 138:1041-1053, 1997; DeZwaan et al., J. Cell Biol. 138:1023-1040, 1997; Gaglio et al., J. Cell Biol. 135:399-414, 1996; Geiser et al., Mol. Biol. Cell 8:1035-1050, 1997; Heck et al., J. Cell Biol. 123:665-679, 1993; Hoyt et al., J. Cell Biol. 118:109-120, 1992; Hoyt et al., Genetics 135:35-44, 1993; Huyett et al., J. Cell Sci. 111:295-301, 1998; Miller et al., Mol. Biol. Cell 9:2051 -2068, 1998; Roof et al., J. Cell Biol. 118:95-108, 1992; Sanders et al., J. Cell Biol. 137:417-431, 1997; Sanders et al., Mol. Biol. Cell 8:1025-0133, 1997; Sanders et al., J. Cell Biol. 128:617-624, 1995; Sanders & Hoyt, Cell 70:451-458, 1992; Sharp et al., J. Cell Biol. 144:125-138, 1999; Straight et al., J. Cell Biol. 143:687-694, 1998; Whitehead et al., J. Cell Sci. 111:2551-2561, 1998; Wilson et al., J. Cell Sci. 110:451-464, 1997).

Mitotic kinesin biological activities include its ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes, such as kinases or proteases; and specific kinesin cellular activities such as spindle pole separation.

Methods for assaying biological activity of a mitotic kinesin are well known in the art. For example, methods of performing motility assays are described, e.g., in Hall et al., Biophys. J., 71:3467-34761996; Turner et al., Anal. Biochem. 242:20-25, 1996; Gittes et al., Biophys. J. 70:418-429, 1996; Shirakawa et al., J. Exp. Biol. 198: 1809-1815, 1995; Winkelmann et al., Biophys. J. 68: 2444-2453, 1995; and Winkelmann et al., Biophys. J. 68:72S, 1995. Methods known in the art for determining ATPase hydrolysis activity also can be used. U.S. Pat. No. 6,410,254 describes such assays. Other methods can also be used. For example, Pᵢ release from kinesin can be quantified. In one embodiment, the ATP hydrolysis activity assay utilizes 0.3 M perchloric acid (PCA) and malachite green reagent (8.27 mM sodium molybdate II, 0.33 mM malachite green oxalate, and 0.8 mM Triton X-100). To perform the assay, 10 μL of reaction is quenched in 90 μL of cold 0.3 M PCA. Phosphate standards are used so data can be converted to nM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to nM Pᵢ and plotted over time. Additionally, ATPase assays known in the art include the luciferase assay.

ATPase activity of kinesin motor domains also can be used to monitor the effects of modulating agents. In one embodiment ATPase assays of kinesin are performed in the absence of microtubules. In another embodiment, the ATPase assays are performed in the presence of microtubules. Different types of modulating agents can be detected in the above assays. In one embodiment, the effect of a modulating agent is independent of the concentration of microtubules and ATP. In another embodiment, the effect of the agents on kinesin ATPase may be decreased by increasing the concentrations of ATP, microtubules, or both. In yet another embodiment, the effect of the modulating agent is increased by increasing concentrations of ATP, microtubules or both.

Agents that reduce the biological activity of a mitotic kinesin in vitro may then be screened in vivo. Methods for in vivo screening include assays of cell cycle distribution, cell viability, or the presence, morphology, activity, distribution, or amount of mitotic spindles. Methods for monitoring cell cycle distribution of a cell population, for example, by flow cytometry, are well known to those skilled in the art, as are methods for determining cell viability (see, e.g., U.S. Pat. No. 6,617,115).

### Mitotic Kinesin Inhibitors

Mitotic kinesin inhibitors include chlorpromazine, monasterol, terpendole E, HR22C16, and SB715992. Other mitotic kinesin inhibitors are those compounds disclosed in Hopkins et al., Biochemistry 39:2805, 2000, Hotha et al., Angew Chem. Inst. Ed. 42:2379, 2003, PCT Publication Nos. WO01/98278, WO02/057244, WO02/079169, WO02/057244, WO02/056880, WO03/050122, WO03/050064, WO03/049679, WO03/049678, WO03/049527, WO03/079973, and WO03/039460, and U.S. Patent Application Publication Nos. 2002/0165240, 2003/0008888, 2003/0127621, and 2002/0143026; and U.S. Pat. Nos. 6,437,115, 6,545,004, 6,562,831, 6,569,853, and 6,630,479, and the chlorpromazine analogs described in U.S. patent application Ser. No. 10/617,424 (see, e.g., Formula (I)).

### Protein Tyrosine Phosphatases

Protein tyrosine phosphatases include the PRL family (PRL-1, PRL-2, and PRL-3), PTP1B, SHP-1, SHP-2, MKP-1, MKP-2, CDC14, CDC25A, CDC25B, CDC25C, PTPα, and PTP-BL. Protein tyrosine phosphatase biological activities include dephosphorylation of tyrosine residues on substrates. The GenBank Accession Nos. of representive tyrosine phosphatases are provided in Table 2.

TABLE 2
Human protein tyrosine phosphatases

| Protein name | GenBank Accession No. |
|---|---|
| PRL-1 | AJ420505, BI222469, U48296 |
| PRL-2 | AF208850, BI552091, L48723 |
| PRL-3 | AF041434, BC003105 |
| PTP1B | AU117677, M33689 |
| SHP-1 | BC002523, BG754792, M77273, BM742181, AF178946 |
| SHP-2 | AU123593, BF515187, BX537632, D13540 |
| MKP-1 | U01669, X68277 |
| MKP-2 | BC014565, U21108, U48807, AL137704 |
| CDC14A | AF000367, AF064102, AF064103 |
| CDC14B | AF023158, AF064104 |
| CDC25A | M81933 |
| CDC25B | M81934, Z68092, AF036233 |
| CDC25C | M34065, Z29077, AJ304504, M34065 |
| PTPα | M36033 |
| PTP-BL | D21210, D21209, D21211, U12128 |

### Protein Tyrosine Phosphatase Inhibitors

Inhibitors of protein tyrosine phosphatases include pentamidine, levamisole, ketoconazole, bisperoxovanadium compounds (e.g., those described in Scrivens et al., Mol. Cancer Ther. 2:1053-1059, 2003, and U.S. Pat. No. 6,642,221), vandate salts and complexes (e.g., sodium orthovanadate), dephosphatin, dnacin A1, dnacin A2, STI-571, suramin, gallium nitrate, sodium stibogluconate, meglumine antimonate, 2-(2-mercaptoethanol)-3-methyl-1,4-naphthoquinone, 2,5-bis(4-amidinophenyl)furan-bis-O-methylamidoxime, known as DB289 (Immtech), 2,5-bis(4-amidinophenyl)furan (DB75, Immtech), disclosed in U.S. Pat. No. 5,843,980, and compounds described in Pestell et al., Oncogene 19:6607-6612, 2000, Lyon et al., Nat. Rev. Drug Discov. 1:961-976, 2002, Ducruet et al., Bioorg. Med. Chem. 8:1451-1466, 2000, U.S. Patent Application Publication Nos. 2003/0114703, 2003/0144338, and 2003/0161893, and PCT Patent Publication Nos. WO99/46237, WO03/06788, and WO03/070158. Still other analogs are those that fall within a formula provided in any of U.S. Pat. Nos. 5,428,051; 5,521,189; 5,602,172; 5,643,935; 5,723,495; 5,843,980; 6,008,247; 6,025,398; 6,172,104; 6,214,883; and 6,326,395, and U.S. Patent Application Publication Nos. 2001/0044468 and 2002/0019437, and the pentamidine analogs described in U.S. patent application Ser. No. 10/617,424 (see, e.g., Formula (II)). Other protein tyrosine phosphatase inhibitors can be identified, for example, using the methods described in Lazo et al. (Oncol. Res. 13:347-352, 2003), PCT Publication Nos. WO97/40379, WO03/003001, and WO03/035621, and U.S. Pat. Nos. 5,443,962 and 5,958,719.

### Other Biological Activity Inhibitors

In addition to reducing biological activity through the use of compounds that bind a mitotic kinesin or protein tyrosine phosphatase, other inhibitors of mitotic kinesin and protein tyrosine phosphatase biological activity can be employed. Such inhibitors include compounds that reduce the amount of target protein or RNA levels (e.g., antisense compounds, dsRNA, ribozymes) and compounds that compete with endogenous mitotic kinesins or protein tyrosine phosphatases for binding partners (e.g., dominant negative proteins or polynucleotides encoding the same).

Antisense Compounds

The biological activity of a mitotic kinesin and/or protein tyrosine phosphatase can be reduced through the use of an antisense compound directed to RNA encoding the target protein. Mitotic kinesin antisense compounds suitable for this use are known in the art (see, e.g., U.S. Pat. No. 6,472,521, WO03/030832, and Maney et al., J. Cell Biol., 1998, 142:787-801), as are antisense compounds against protein tyrosine phosphatases (see, e.g., U.S. Patent Publication No. 2003/0083285 and Weil et al., Biotechniques 33:1244, 2002). Other antisense compounds that reduce mitotic kinesins can be identified using standard techniques. For example, accessible regions of the target mitotic kinesin or protein tyrosine phosphatase mRNA can be predicted using an RNA secondary structure folding program such as MFOLD (M. Zuker, D. H. Mathews & D. H. Turner, Algorithms and Thermodynamics for RNA Secondary Structure Prediction: A Practical Guide. In: RNA Biochemistry and Biotechnology, J. Barciszewski & B. F. C. Clark, eds., NATO ASI Series, Kluwer Academic Publishers, (1999)). Sub-optimal folds with a free energy value within 5% of the predicted most stable fold of the mRNA are predicted using a window of 200 bases within which a residue can find a complimentary base to form a base pair bond. Open regions that do not form a base pair are summed together with each suboptimal fold and areas that are predicted as open are considered more accessible to the binding to antisense nucleobase oligomers. Other methods for antisense design are described, for example, in U.S. Pat. No. 6,472,521, Antisense Nucleic Acid Drug Dev. 1997 7:439-444, Nucleic Acids Res. 28:2597-2604, 2000, and Nucleic Acids Research 31:4989-4994, 2003.

RNA Interference

The biological activity of a mitotic kinesin and/or protein tyrosine phosphatase can be reduced through the use of RNA interference (RNAi), employing, e.g., a double stranded RNA (dsRNA) or small interfering RNA (siRNA) directed to the mitotic kinesin or protein tyrosine phosphatase in question (see, e.g., Miyamoto et al., Prog. Cell Cycle Res. 5:349-360, 2003; U.S. Patent Application Publication No. 2003/0157030). Methods for designing such interfering RNAs are known in the art. For example, software for designing interfering RNA is available from Oligoengine (Seattle, Wash.).

Dominant Negative Proteins

One skilled in the art would know how to make dominant negative mitotic kinesins and protein tyrosine phosphatases. Such dominant negative proteins are described, for example, in Gupta et al., J. Exp. Med., 186:473-478, 1997; Maegawa et al., J. Biol. Chem. 274:30236-30243, 1999; and Woodford-Thomas et al., J. Cell Biol. 117:401-414, 1992.

### Aurora Kinase Inhibitors

Aurora kinases have been shown to be protein kinases of a new family that regulate the structure and function of the mitotic spindle. One target of Aurora kinases include mitotic kinesins. Aurora kinase inhibitors thus can be used in combination with a compound that reduces protein tyrosine phosphatase biological activity according to a method, composition, or kit of the invention.

There are three classes of aurora kinases: aurora-A, aurora-B and aurora-C. Aurora-A includes AIRK1, DmAurora, HsAurora-2, HsAIK, HsSTK15, CeAIR- 1, MmARK1, MmAYK1, MMIAK1 and XIEg2. Aurora-B includes AIRK-2, DmIAL-1, HsAurora-1, HsAIK2, HsAIM-1, HsSTK12, CeAIR-2, MmARK2 and XAIRK2. Aurora-C includes HsAIK3 (Adams, et al., Trends Cell Biol. 11:49-54, 2001).

Aurora kinase inhibitors include VX-528 and ZM447439; others are described, e.g., in U.S. Patent Application Publication No. 2003/0105090 and U.S. Pat. Nos. 6,610,677, 6,593,357, and 6,528,509.

### Farnesyltransferase Inhibitors

Farnesyltransferase inhibitors alter the biological activity of PRL phosphatases and thus can be used in combination with a compound that reduces mitotic kinesin activity in a method, composition, or kit of the invention. Farnesyltransferase inhibitors include arglabin, lonafarnib, BAY-43-9006, tipifarnib, perillyl alcohol, FTI-277, and BMS-214662, as well as those compounds described, e.g., in Kohl, Ann. NY Acad. Sci. 886:91-102, 1999, U.S. Patent Application Publication Nos. 2003/0199544, 2003/0199542, 2003/0087940, 2002/0086884, 2002/0049327, and 2002/0019527, U.S. Pat. Nos. 6,586,461 and 6,500,841, and WO03/004489.

### Therapy

The compounds of the invention are useful for the treatment of cancers and other disorders characterized by hyperproliferative cells. Therapy may be performed alone or in conjunction with another therapy (e.g., surgery, radiation therapy, chemotherapy, immunotherapy, anti-angiogenesis therapy, or gene therapy). Additionally, a person having a greater risk of developing a neoplasm or other proliferative disease (e.g., one who is genetically predisposed or one who previously had such a disorder) may receive prophylactic treatment to inhibit or delay hyperproliferation. The duration of the combination therapy depends on the type of disease or disorder being treated, the age and condition of the patient, the stage and type of the patient's disease, and how the patient responds to the treatment. Therapy may be given in on-and-off cycles that include rest periods so that the patient's body has a chance to recovery from any as yet unforeseen side-effects. Desirably, the methods, compositions, and kits of the invention are more effective than other methods, compositions, and kits. By “more effective” is meant that a method, composition, or kit exhibits greater efficacy, is less toxic, safer, more convenient, better tolerated, or less expensive, or provides more treatment satisfaction than another method, composition, or kit with which it is being compared.

Cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

Other proliferative disease that can be treated with the combinations and methods of the invention include lymphoproliferative disorders and psoriasis. By “lymphoproliferative disorder” is meant a disorder in which there is abnormal proliferation of cells of the lymphatic system (e.g., T-cells and B-cells), and includes multiple sclerosis, Crohn's disease, lupus erythematosus, rheumatoid arthritis, and osteoarthritis.

### EXAMPLES

The following examples are to illustrate the invention. They are not meant to limit the invention in any way.

### Chlorpromazine is a Mitotic Kinesin Inhibitor

We determined that chlorpromazine is a mitotic kinesin inhibitor using a cell free motor assay. This assay measures organic phosphate (Pᵢ) generated during microtubule activated ATPase activity of kinesin motor proteins. Recombinant HsEg5/KSP kinesin motor protein activity was assayed using the Kinesin ATPase End Point Biochem Kit (Cytoskeleton, catalog #BK053) following the manufacturer's instructions for amounts of reaction buffer, ATP and microtubules. The amount of HsEg5/KSP kinesin protein was optimized to 0.8 μg per reaction and included where indicated. Each assay was performed in a total reaction volume of 30 μL in a clear 96 well ½ area plate (Corning Inc., Costar and cat #3697) and included the following conditions:
1. a reaction blank consisting of reaction buffer and ATP only;
2. negative control reactions containing:
   a. microtubules and ATP without kinesin protein or
   b. kinesin HsEg5/KSP and ATP without microtubules; and
3. experimental reactions containing ATP, kinesin, and microtubules with or without compound at the indicated final concentrations.

Reactions were pre-incubated for 15 minutes at room temperature prior to the addition of ATP. After ATP addition, reactions were allowed to proceed for 10 minutes at room temperature prior to termination by the addition of 70 μL of CytoPhos Reagent. Following a last 10-minute incubation at room temperature, reactions were quantitated by reading the absorbance at 650 nm on a spectrophotometer (Beckman Instruments, Inc., Model DU 530). Raw absorbance values were corrected by subtracting the absorbance of the blank. Absorbance was converted into Pi concentration by comparison with a standard Pi curve. Percent inhibition was calculated from Pi concentration according to the following formula: %Inhibition=(untreated−treated)/untreated×100. The arithmetic mean was generated from percent inhibition of experimental replicates. The results are shown in Table 4.

TABLE 4
Percent inhibition of kinesin motor activity (n = 4)

| | Chlorpromazine [μM] | | | | | | |
|---|---|---|---|---|---|---|---|
| | 1 | 2 | 4 | 8 | 16 | 32 | 64 |
| Mean | −5.51 | −11.18 | 17.42 | 52.91 | 85.82 | 97.79 | 104.54 |
| STDEV | 11.87 | 25.94 | 17.54 | 6.99 | 10.84 | 6.40 | 10.96 |

Other phenothiazines capable of reducing mitotic kinesin biological activity include promethazine, thioridazine, trifluoperazine, perphenazine, fluphenazine, clozapine, and prochlorperazine.

### The Combination of Chlorpromazine and Pentamidine Reduce Cell Proliferation in Vitro

The ability of pentamidine (a protein tyrosine phosphatase inhibitor) and chlorpromazine (a mitotic kinesin inhibitor), in combination, to reduce cell proliferation in vitro was determined. Human colon adenocarcinoma cell line HCT116 (ATCC#CCL-247) were grown at 37°±5° C. and 5% CO₂ in DMEM supplemented with 10% FBS, 2 mM glutamine, 1% penicillin and 1% streptomycin. The anti-proliferation assays were performed in 384-well plates. 10× stock solutions (6.6 μL) from the combination matrices were added to 40 μL of culture media in assay wells. The tumor cells were liberated from the culture flask using a solution of 0.25% trypsin. Cells were diluted in culture media such that 3000 cells were delivered in 20 μL of media into each assay well. Assay plates were incubated for 72-80 hours at 37° C.±0.5° C. with 5% CO2. Twenty microliters of 20% Alamar Blue warmed to 37° C.±0.5° C. was added to each assay well following the incubation period. Alamar Blue metabolism was quantified by the amount of fluorescence intensity 3.5-5.0 hours after addition. Quantification, using an LJL Analyst AD reader (LJL Biosystems), was taken in the middle of the well with high attenuation, a 100 msec read time, an excitation filter at 530 nm, and an emission filter at 575 nm. For some experiments, quantification was performed using a Wallac Victor2 reader. Measurements were taken at the top of the well with stabilized energy lamp control; a 100 msec read time, an excitation filter at 530 nm, and an emission filter at 590 nm. No significant differences between plate readers were measured.

The percent inhibition (%I) for each well was calculated using the following formula:
%I=[(avg. untreated wells−treated well)/(avg. untreated wells)]×100

The average untreated well value (avg. untreated wells) is the arithmetic mean of 40 wells from the same assay plate treated with vehicle alone. Negative inhibition values result from local variations in treated wells as compared to untreated wells. The data, expressed as percent inhibition, are shown in Table 5.

TABLE 5

| | | Chlorpromazine (μM) | | | | | | | | | |
|---|---|---|---|---|---|---|---|---|---|---|---|
| | | 0 | 4 | 6 | 7.5 | 9 | 10 | 12 | 16 | 20 | 22 |
| Pentamidine | 0 | 0.63 | 2.9 | 0.11 | 5.4 | 4.1 | 16 | 22 | 39 | 56 | 59 |
| (μM) | 0.5 | 1.2 | −0.13 | 6.1 | 4.3 | 7.9 | 16 | 31 | 45 | 64 | 65 |
| | 1 | 1.9 | 2.2 | 9.1 | 5.5 | 16 | 21 | 25 | 56 | 57 | 68 |
| | 2 | 3.1 | 3.1 | 5.8 | 5.1 | 9.7 | 18 | 30 | 57 | 70 | 73 |
| | 4 | −0.77 | 4.0 | 2.7 | 12 | 10 | 20 | 26 | 59 | 69 | 74 |
| | 6 | 5 | 7.1 | 15 | 9.9 | 16 | 22 | 38 | 58 | 74 | 78 |
| | 9 | 9 | 13 | 13 | 22 | 16 | 37 | 41 | 68 | 79 | 88 |
| | 12 | 9.9 | 13 | 15 | 16 | 18 | 27 | 46 | 69 | 82 | 87 |
| | 15 | 16 | 20 | 22 | 35 | 26 | 40 | 52 | 78 | 84 | 92 |
| | 20 | 19 | 22 | 25 | 36 | 40 | 49 | 70 | 82 | 94 | 94 |

### OTHER EMBODIMENTS

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in oncology or related fields are intended to be within the scope of the invention.

1. A method for identifying a combination that may be useful for the treatment of a proliferative disease, the method comprising the steps of:
(a) contacting proliferating cells in vitro with an agent that reduces mitotic kinesin biological activity and a candidate compound; and
(b) determining whether the combination of the agent and the candidate compound reduces cell proliferation, relative to proliferation of cells contacted with the agent but not contacted with the candidate compound,
wherein a reduction in cell proliferation identifies the combination as a combination that may be useful for the treatment of a proliferative disease.

2. The method of claim 1, wherein said agent that reduces mitotic kinesin biological activity is a mitotic kinesin inhibitor.

3. The method of claim 1, wherein said agent that reduces mitotic kinesin biological activity is an antisense compound or RNAi compound that reduces the expression levels of said mitotic kinesin.

4. The method of claim 1, wherein said agent that reduces mitotic kinesin biological activity is a dominant negative mitotic kinesin or an expression vector encoding said dominant negative mitotic kinesin.

5. The method of claim 1, wherein said agent that reduces mitotic kinesin biological activity is an antibody that binds said mitotic kinesin and reduces mitotic kinesin biological activity.

6. The method of claim 1, wherein said mitotic kinesin is HsEg5/KSP.

7. The method of claim 1, wherein said agent that reduces mitotic kinesin biological activity in an aurora kinase inhibitor.

8. The method of claim 1, wherein said mitotic kinesin biological activity is enzymatic activity, motor activity, or binding activity.

9. The method of claim 1, wherein the cells are cancer cells or cells from a cancer cell line.

10. A method for identifying a compound that may be useful for the treatment of a proliferative disease, the method comprising the steps of:
(a) providing proliferating cells engineered to have reduced mitotic kinesin biological activity;
(b) contacting the cells with a candidate compound; and
(c) determining whether the candidate compound reduces cell proliferation, relative to cells not contacted with the candidate compound,
wherein a reduction in cell proliferation identifies the compound as a compound that may be useful for the treatment of a proliferative disease.

11. The method of claim 10, wherein the cells are cancer cells or cells from a cancer cell line.

12. A method for identifying a combination that may be useful for the treatment of a proliferative disease, the method comprising the steps of:
(a) contacting proliferating cells in vitro with an agent that reduces protein tyrosine phosphatase biological activity and a candidate compound; and
(b) determining whether the combination of the agent and the candidate compound reduces cell proliferation, relative to proliferation of cells contacted with the agent but not contacted with the candidate compound,
wherein a reduction in cell proliferation identifies the combination as a combination that may be useful for the treatment of a proliferative disease.

13. The method of claim 12, wherein said agent that reduces protein tyrosine phosphatase biological activity is a protein tyrosine phosphatase inhibitor.

14. The method of claim 12, wherein said agent that reduces protein tyrosine phosphatase biological activity is an antisense compound or RNAi compound that reduces the expression levels of said protein tyrosine phosphatase.

15. The method of claim 12, wherein said agent that reduces protein tyrosine phosphatase biological activity is a dominant negative protein tyrosine phosphatase or an expression vector encoding said dominant negative protein tyrosine phosphatase.

16. The method of claim 12, wherein said agent that reduces protein tyrosine phosphatase biological activity is an antibody that binds said protein tyrosine phosphatase and reduces protein tyrosine phosphatase biological activity.

17. The method of claim 12, wherein said protein tyrosine phosphatase is PTP1B, PRL-1, PRL-2, PRL-3, SHP-1, SHP-2, MKP-1, MKP-2, CDC14, CDC25A, CDC25B, or CDC25C.

18. The method of claim 12, wherein said second agent is a farnesyltransferase inhibitor.

19. The method of claim 12, wherein the cells are cancer cells or cells from a cancer cell line.

20. A method for identifying a compound that may be useful for the treatment of a proliferative disease, the method comprising the steps of:
(a) providing proliferating cells engineered to have reduced protein tyrosine phosphatase biological activity;
(b) contacting the cells with a candidate compound; and
(c) determining whether the candidate compound reduces cell proliferation, relative to cells not contacted with the candidate compound,
wherein a reduction in cell proliferation identifies the compound as a compound that may be useful for the treatment of a proliferative disease.

21. A method for identifying a combination that may be useful for the treatment of a proliferative disease, the method comprising the steps of:
(a) identifying a compound that reduces mitotic kinesin biological activity;
(b) contacting proliferating cells in vitro with an agent that reduces protein tyrosine phosphatase biological activity and the compound identified in step (a); and
(c) determining whether the combination of the agent and the compound identified in step (a) reduces cell proliferation, relative to proliferation of cells contacted with the agent but not contacted with the compound identified in step (a) or contacted with the compound identified in step (a) but not contacted with the agent,
wherein a reduction in cell proliferation identifies the combination as a combination that may be useful for the treatment of a proliferative disease.

22. A method for identifying a combination that may be useful for the treatment of a proliferative disease, the method comprising the steps of:
(a) identifying a compound that reduces protein tyrosine phosphatase biological activity;
(b) contacting proliferating cells in vitro with an agent that reduces mitotic kinesin biological activity and the compound identified in step (a); and
(c) determining whether the combination of the agent and the compound identified in step (a) reduces cell proliferation, relative to proliferation of cells contacted with the agent but not contacted with the compound identified in step (a) or contacted with the compound identified in step (a) but not contacted with the agent,
wherein a reduction in cell proliferation identifies the combination as a combination that may be useful for the treatment of a proliferative disease.
